SEQUENCE LISTING (4 sequences)

SEQ ID NO: 1 - DNA, 1305 bp, Artificial Sequence
  ICL1
atgaaaacccgtacacaacaaattgaagaattacagaaagagtggactcaaccgcgttgggaaggcattactcgcccatacagtgcggaagatgtggtgaaattacgcggttcagtcaatcctgaatgcacgctggcgcaactgggcgcagcgaaaatgtggcgtctgctgcacggtgagtcgaaaaaaggctacatcaacagcctcggcgcactgactggcggtcaggcgctgcaacaggcgaaagcgggtattgaagcagtctatctgtcgggatggcaggtagcggcggacgctaacctggcggccagcatgtatccggatcagtcgctctatccggcaaactcggtgccagctgtggtggagcggatcaacaacaccttccgtcgtgccgatcagatccaatggtccgcgggcattgagccgggcgatccgcgctatgtcgattacttcctgccgatcgttgccgatgcggaagccggttttggcggtgtcctgaatgcctttgaactgatgaaagcgatgattgaagccggtgcagcggcagttcacttcgaagatcagctggcgtcagtgaagaaatgcggtcacatgggcggcaaagttttagtgccaactcaggaagctattcagaaactggtcgcggcgcgtctggcagctgacgtgacgggcgttccaaccctgctggttgcccgtaccgatgctgatgcggcggatctgatcacctccgattgcgacccgtatgacagcgaatttattaccggcgagcgtaccagtgaaggcttcttccgtactcatgcgggcattgagcaagcgatcagccgtggcctggcgtatgcgccatatgctgacctggtctggtgtgaaacctccacgccggatctggaactggcgcgtcgctttgcacaagctatccacgcgaaatatccgggcaaactgctggcttataactgctcgccgtcgttcaactggcagaaaaacctcgacgacaaaactattgccagcttccagcagcagctgtcggatatgggctacaagttccagttcatcaccctggcaggtatccacagcatgtggttcaacatgtttgacctggcaaacgcctatgcccagggcgagggtatgaagcactacgttgagaaagtgcagcagccggaatttgccgccgcgaaagatggctataccttcgtatctcaccagcaggaagtgggtacaggttacttcgataaagtgacgactattattcagggcggcacgtcttcagtcaccgcgctgaccggctccactgaagaatcgcagttctaa

SEQ ID NO: 2 - DNA, 1959 bp, Artificial Sequence
  ACS1
atgagccaaattcacaaacacaccattcctgccaacatcgcagaccgttgcctgataaaccctcagcagtacgaggcgatgtatcaacaatctattaacgtacctgataccttctggggcgaacagggaaaaattcttgactggatcaaaccttaccagaaggtgaaaaacacctcctttgcccccggtaatgtgtccattaaatggtacgaggacggcacgctgaatctggcggcaaactgccttgaccgccatctgcaagaaaacggcgatcgtaccgccatcatctgggaaggcgacgacgccagccagagcaaacatatcagctataaagagctgcaccgcgacgtctgccgcttcgccaataccctgctcgagctgggcattaaaaaaggtgatgtggtggcgatttatatgccgatggtgccggaagccgcggttgcgatgctggcctgcgcccgcattggcgcggtgcattcggtgattttcggcggcttctcgccggaagccgttgccgggcgcattattgattccaactcacgactggtgatcacttccgacgaaggtgtgcgtgccgggcgcagtattccgctgaagaaaaacgttgatgacgcgctgaaaaacccgaacgtcaccagcgtagagcatgtggtggtactgaagcgtactggcgggaaaattgactggcaggaagggcgcgacctgtggtggcacgacctggttgagcaagcgagcgatcagcaccaggcggaagagatgaacgccgaagatccgctgtttattctctacacctccggttctaccggtaagccaaaaggtgtgctgcatactaccggcggttatctggtgtacgcggcgctgacctttaaatatgtctttgattatcatccgggtgatatctactggtgcaccgccgatgtgggctgggtgaccggacacagttacttgctgtacggcccgctggcctgcggtgcgaccacgctgatgtttgaaggcgtacccaactggccgacgcctgcccgtatggcgcaggtggtggacaagcatcaggtcaatattctctataccgcacccacggcgatccgcgcgctgatggcggaaggcgataaagcgatcgaaggcaccgaccgttcgtcgctgcgcattctcggttccgtgggcgagccaattaacccggaagcgtgggagtggtactggaaaaaaatcggcaacgagaaatgtccggtggtcgatacctggtggcagaccgaaaccggcggtttcatgatcaccccgctgcctggcgctaccgagctgaaagccggttcggcaacacgtccgttcttcggcgtgcaaccggcgctggtcgataacgaaggtaacccgctggagggggccaccgaaggtagcctggtaatcaccgactcctggccgggtcaggcgcgtacgctgtttggcgatcacgaacgttttgaacagacctacttctccaccttcaaaaatatgtatttcagcggcgacggcgcgcgtcgcgatgaagatggctattactggataaccgggcgtgtggacgacgtgctgaacgtctccggtcaccgtctggggacggcagagattgagtcggcgctggtggcgcatccgaagattgccgaagccgccgtagtaggtattccgcacaatattaaaggtcaggcgatctacgcctacgtcacgcttaatcacggggaggaaccgtcaccagaactgtacgcagaagtccgcaactgggtgcgtaaagagattggcccgctggcgacgccagacgtgctgcactggaccgactccctgcctaaaacccgctccggcaaaattatgcgccgtattctgcgcaaaattgcggcgggcgataccagcaacctgggcgatacctcgacgcttgccgatcctggcgtagtcgagaagctgcttgaagagaagcaggctatcgcgatgccatcgtaa

SEQ ID NO: 3 - DNA, 1602 bp, Artificial Sequence
  MLS1
atgactgaacaggcaacaacaaccgatgaactggctttcacaaggccgtatggcgagcaggagaagcaaattcttactgccgaagcggtagaatttctgactgagctggtgacgcattttacgccacaacgcaataaacttctggcagcgcgcattcagcagcagcaagatattgataacggaacgttgcctgattttatttcggaaacagcttccattcgcgatgctgattggaaaattcgcgggattcctgcggacttagaagaccgccgcgtagagataactggcccggtagagcgcaagatggtgatcaacgcgctcaacgccaatgtgaaagtctttatggccgatttcgaagattcactggcaccagactggaacaaagtgatcgacgggcaaattaacctgcgtgatgcggttaacggcaccatcagttacaccaatgaagcaggcaaaatttaccagctcaagcccaatccagcggttttgatttgtcgggtacgcggtctgcacttgccggaaaaacatgtcacctggcgtggtgaggcaatccccggcagcctgtttgattttgcgctctatttcttccacaactatcaggcactgttggcaaagggcagtggtccctatttctatctgccgaaaacccagtcctggcaggaagcggcctggtggagcgaagtcttcagctatgcagaagatcgctttaatctgccgcgcggcaccatcaaggcgacgttgctgattgaaacgctgcccgccgtgttccagatggatgaaatccttcacgcgctgcgtgaccatattgttggtctgaactgcggtcgttgggattacatcttcagctatatcaaaacgttgaaaaactatcccgatcgcgtcctgccagacagacaggcagtgacgatggataaaccattcctgaatgcttactcacgcctgttgattaaaacctgccataaacgcggtgcttttgcgatgggcggcatggcggcgtttattccgagcaaagatgaagagcacaataaccaggtgctcaacaaagtaaaagcggataaatcgctggaagccaataacggtcacgatggcacatggatcgctcacccaggccttgcggacacggcaatggcggtattcaacgacattctcggctcccgtaaaaatcagcttgaagtgatgcgcgaacaagacgcgccgattactgccgatcagctgctggcaccttgtgatggtgaacgcaccgaagaaggtatgcgcgccaacattcgcgtggctgtgcagtacatcgaagcgtggatctctggcaacggctgtgtgccgatttatggcctgatggaagatgcggcgacggctgaaatttcccgtacctcgatctggcagtggatccatcatcaaaaaacgttgagcaatggcaaaccggtgaccaaagccttgttccgccagatgctgggcgaagagatgaaagtcattgccagcgaactgggcgaagaacgtttctcccaggggcgttttgacgatgccgcacgcttgatggaacagatcaccacttccgatgagttaattgatttcctgaccctgccaggctaccgcctgttagcgtaa

SEQ ID NO: 4 - DNA, 939 bp, Artificial Sequence
  MDH3
atgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcgcgtaaaccgggtatggatcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

(19) United States
(12) Patent Application Publication
(10) Pub. No.: US 2011/0300595 A1
(43) Pub. Date: Dec. 8, 2011

(54) MICROORGANISM FOR PRODUCING SUCCINIC ACID

(75) Inventors: Christine Lang, Berlin (DE); Andreas Raab, Berlin (DE)

(73) Assignee: ORGANO BALANCE GMBH, Berlin (DE)

(21) Appl. No.: 13/124,436

(22) PCT Filed: Oct. 7, 2009
(86) PCT No.: PCT/DE2009/001386

§ 371 Date: Aug. 18, 2011

(30) Foreign Application Priority Data
Oct. 17, 2008 (DE) 10 2008 051 727.5

(51) Int. Cl. C12P 7/46; C12N 1/19

(52) U.S. Cl. 435/145; 435/254.2; 435/254.21; 435/254.22; 435/254.23

(57) ABSTRACT

The invention relates to an isolated, genetically modified microorganism, wherein compared to the wild type a) the idh1 and idp1 genes have been deleted or inactivated, and/or b) the sdh2 and sdh1 genes have been deleted or inactivated, and/or c) the PDC2 gene has been deleted or inactivated or is under the control of a promoter which can be suppressed or induced by exposure of the microorganism using an inductor substance, and/or d) one or more genes from the group consisting of ICL1, MLS1, ACS1 and MDH3 has been replaced or supplemented by a corresponding foreign gene or corresponding foreign genes from Crabtree-negative organisms, and to the uses thereof.

### FIELD THE INVENTION

The invention relates to a microorganism, which compared to the wild type is genetically modified and which is suitable for producing organic acids, in particular succinic acid, to the uses of such a microorganism and to methods for producing such a microorganism.

### PRIOR ART AND BACKGROUND OF THE INVENTION

Dicarboxylic acids have a great economic potential, since they can be used as precursor substances for many chemicals. For instance, succinic acid serves as a precursor for producing plastic materials based on 1,4-butanediol, tetrahydrofuran and gamma-butyrolactone. Today, succinic acid is chemically produced by catalytic hydration of maleic acid anhydride to succinic acid anhydride and subsequent water addition or by direct catalytic hydration of maleic acid.

Succinic acid is also generated by many microorganisms based on sugars or amino acids under physiological environmental conditions. Under anaerobic conditions, usually, besides succinic acid, further fermentation end products such as ethanol, lactic acid, acetic acid and formic acid are generated. The biosynthesis of succinic acid with its high oxygen content requires a reductive CO₂ fixation.

Succinic acid is a metabolite that is normally enriched by anaerobic fermentation processes. Whereas the yield and the enrichment of the product under anaerobic conditions is many times better than under aerobic conditions, the drawback of an exclusively anaerobic process is a technical limitation of the biomass production and a low productivity of the microbial producer. Thus, the consequence is a relatively low biomass/product efficiency. Further, it is difficult to technically handle strictly anaerobic microorganisms.

Different microorganisms that are capable of synthesizing succinic acid under anaerobic conditions are known in the art. The document U.S. Pat. No. 5,143,834 describes a variant of A. succiniciprofucens. It is an obligate anaerobic microorganism that can produce small amounts only of succinic acid and moreover is not capable of tolerating high osmotic pressures and salt concentrations.

The document U.S. Pat. No. 7,063,968 describes a microbial isolate from cattle rumen, Mannheimia sp. 55E, which is capable of synthesizing organic acids under aerobic as well as anaerobic conditions. This is however not a specific enrichment of succinic acid, but a mixture of different organic acids, such as formic acid, acetic acid, lactic acid and succinic acid. The drawback of this producer is that an economic use of the strain is only possible under difficulties, if not even impossible, since for obtaining succinic acid expensive enrichment and purification methods would have to be applied.

The document U.S. Pat. No. 5,869,301 describes a method for producing dicarboxylic acids in a two-step fermentation process with E. coli AFP-111, wherein in a first phase microbial biomass is produced under aerobic conditions and in a second phase the production of succinic acid is carried out anaerobically. The first phase of the generation of biomass is limited in this process, since the glucose concentration in the fed-batch process must be limited to 1 g/L, in order to avoid an enrichment of acetate that would disturb the process generating biomass as well as producing succinic acid. Thus the generation of biomass by this process is possible to a limited extent only. Furthermore, the biosynthesis pathway for the succinic acid is in this process subject to a strong catabolite repression, since genes of the glycolysis, of the citrate cycle and of the glyoxylate pathway are strongly suppressed by glucose, as known from the document (DeRisi et al. 1997). The consequence is that the synthesis of succinic acid in presence of glucose is largely suppressed and thus is strongly limited.

From the document U.S. Pat. No. 6,190,914, microorganisms are known in the art, in which by modulation of suitable transcription factors and kinases the glucose repression of various genes is reduced. The production of organic acids by means of such microorganisms, however in particular also of microorganisms being optimized for the production of organic acids, is not mentioned therein.

### TECHNICAL OBJECT OF THE INVENTION

It is the technical object of the invention to specify a microorganism, by which an improved yield of organic acids, in particular succinic acid, can be obtained in microbiological production methods.

### BASICS OF THE INVENTION AND PREFERRED EMBODIMENTS

For achieving this technical object, the invention teaches an isolated genetically modified microorganism, wherein compared to the wild type a) the genes idh1 and idp1 have been deleted or inactivated, and/or b) the genes sdh2 and sdh1 have been deleted or inactivated, and/or c) the gene PDC2 has been deleted or inactivated or is under the control of a promoter which can be suppressed or induced by exposure of the microorganism using an inductor substance, and/or d) one or more genes from the group consisting of ICL1, MLS1, ACS1 and MDH3 has been replaced or supplemented by a corresponding foreign gene or corresponding foreign genes from a Crabtree-negative organism.

The foreign gene, which replaces or supplements the gene ICL1, may have at least 75% homology to sequence No. 1. The foreign gene, which replaces or supplements the gene ACS1, may have at least 75% homology to sequence No. 2. The foreign gene, which replaces or supplements the gene MLS1, may have at least 75% homology to sequence No. 3. The foreign gene, which replaces or supplements the gene MDH3, may have at least 75% homology to sequence No. 4. Preferably the homology is more than 80%, in particular more than 90%, most preferably more than 95%. It may however also be identical therewith.

By the invention, an optimized method for producing succinic acid and other organic acids of the respiratory central metabolism by means of a yeast strain, in particular of a Saccharomyces cerevisiae yeast strain, is obtained. This method permits a more efficient production of organic carboxylic acids, in particular dicarboxylic acids and hydroxy fatty acids of the respiratory central metabolism of the yeast, such as for instance succinic acid, with regard to the production time and yields. Furthermore, it allows a 2-step production process by separation of growth and production phases without the use of an antibiotic-dependent promoter system.

By the invention, a process is made possible, in which initially in a first growth phase, biomass is enriched and in a second phase, succinic acid is produced and transferred into the culture medium. The separation of growth and production phases contributes to a large extent to the efficiency of the complete production process of organic acids in yeast, since growth of the cells and production of the desired metabolites are otherwise always competing factors.

During the production phase, the generation of biomass is undesired, since carbon or substrate used therefor is consumed, which ultimately will lead to a reduction of the yield of the metabolites to be produced, for instance succinic acid.

The separation of growth and production phases can be achieved by means of a microorganism modified genetically or by genetic mutation and an associated fermentation method, in which initially during a first phase an optimum biomass increase of the microbial producer is secured and then, in a second phase (production phase), follows the enrichment of carboxylic acids, for instance succinic acid, from primary carbon sources (e.g., glucose) and CO₂ (anaplerotic reaction, CO₂ fixation).

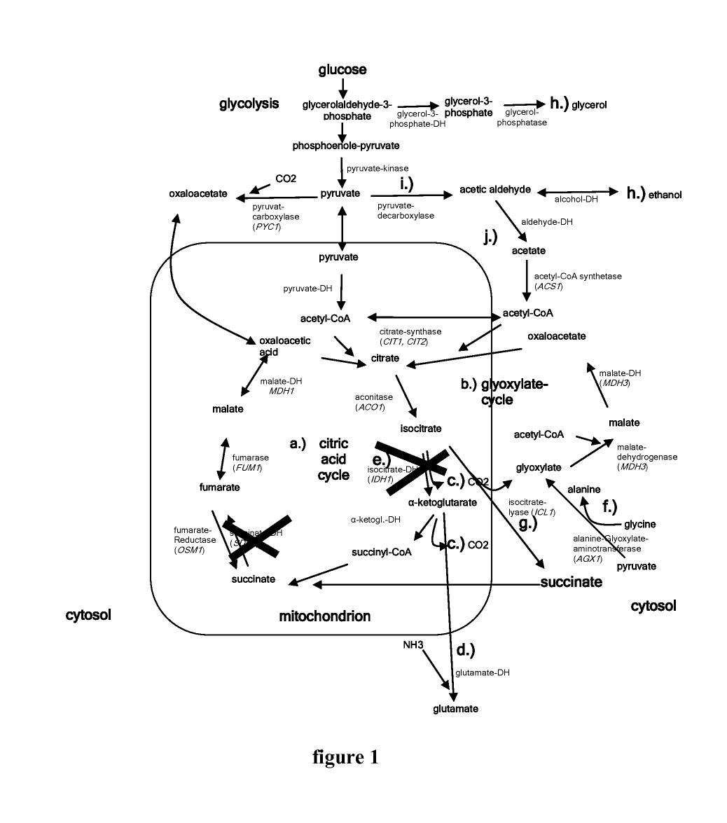

During the production phase, by genetic modification of a microorganism, which is described in the not pre-published patent application PCT/DE 2008/000670 “Microorganism for producing succinic acid”, the citrate cycle (therein FIG. 1a.)) is interrupted after the intermediates isocitrate and succinate (therein in FIG. 1 marked by black crosses), whereas it is not interrupted in the growth phase and has the configuration of the wild type. These interruptions lead to that succinate cannot be further metabolized and is enriched as an end product and the carbon flow is redirected into the glyoxylate cycle (therein FIG. 1b.)). For the production of organic acids by the glyoxylate cycle, there is the advantage that the two oxidative decarboxylation steps of isocitrate to succinyl-CoA in the citrate cycle, and thus the loss of carbon in the form of twice CO₂ (see therein FIG. 1c.)) is prevented. The described interruptions after the intermediates isocitrate and succinate during the production phase are achieved by an exogenously controllable promoter system, which is connected upstream of the genes to be suppressed in the production phase. The repression of the corresponding genes during the production phase takes then place by addition of a tetracyclic antibiotic, for instance tetracycline, to the culture medium. The application of antibiotics in the fermentation may however lead to problems, since they are an additional cost factor. Furthermore, antibiotics need if applicable to be removed again from the product or the culture broth, which makes the “downstream processing” considerably more expensive and more cost-intensive.

In contrast, the invention provides another optimized possibility of the separation of growth and production phases, which does not require the application of antibiotics, as well as an optimized method for producing succinic acid or other organic acids in yeast.

The following observations must be made concerning feature a). For instance in the yeast Saccharomyces cerevisiae, in spite of the deletions of the genes sdh2 and idh1, which lead to an interruption of the citrate cycle, a growth rate can be measured that is comparable to that of an unmodified wild type yeast. A growth of a yeast strain with an idh1 deletion is only possible, since this deletion does not lead to a complete disappearance of the isocitrate dehydrogenase activity. The reason for this is the existence of three further isoenzymes of the isocitrate dehydrogenase, which can compensate the loss of the dimeric main enzyme, coded by the genes IDH1 and IDH2, with respect to the generation of α-ketoglutarate. The synthesis of α-ketoglutarate is absolutely necessary for a growth of the yeast cell on minimal media, since from this intermediate the amino acid glutamate is generated, without which no growth is possible. In a 2-step fermentation process for producing succinic acid with yeast, the effective separation of growth and production phases is thus only possible by the complete inhibition of the isocitrate dehydrogenase activity in the production strain, since thereby a glutamate auxotrophy is secured, with the consequence that the yeast in medium without supplemented glutamate has no growth. This can be used in order to control the fermentation process via the supplementation of glutamate to the culture medium. By the quantity of the added glutamate to the culture medium, the duration of the growth phase and the intended cell density can effectively be controlled in this phase. With increasing quantity, duration and cell density will also increase. When the glutamate in the culture medium is consumed, no further growth is possible and all carbon can effectively be used for the synthesis of succinic acid, the generation of which is then not in competition to the generation of biomass. This separation of growth and production phases in the fermentation process by the supplementation of glutamate is achieved, if in addition to the deletion of the gene idh1 at least also the gene idp1 (Contreras-Shannon et al. 2005), preferably also the genes idp2 and idp3 coding for isoenzymes of the isocitrate dehydrogenase are deleted. A practically complete inhibition of the isocitrate dehydrogenase activity secures the glutamate auxotrophy required therefor. Another advantage of the complete inhibition of the isocitrate dehydrogenase activity is that thus all carbon in the respiratory system of the yeast is redirected into the glyoxylate cycle in the direction of succinic acid and cannot flow off to α-ketoglutarate, which would lead to yield losses.

The following observations must be made concerning feature b). In order to avoid or reduce further yield losses, succinic acid should be enriched as an end product and should not further be metabolized by the yeast cell. This cannot be achieved by the singular deletion of the gene sdh2. In addition, according to the invention, another subunit of the heterotetrameric enzyme succinate dehydrogenase, coded by the gene sdh1, is deleted. (Kubo et al. 2000) detected a residual activity of the succinate dehydrogenase in a yeast strain with an sdh2 deletion, which was inhibited by additional deletion of the gene sdh1. Yield losses may also result by the further metabolization of the generated succinate via the enzyme succinate-semialdehyde dehydrogenase. This enzyme is part of the glutamate degradation pathway and catalyzes the reaction of succinate to succinate-semialdehyde. This intermediate is then metabolized by gamma-amino butyric acid to glutamate. In this way, not only yield losses may occur, but α-ketoglutarate and glutamate may also be synthesized, which makes a control of the fermentation process by glutamate supplementation impossible. Therefore, the additional deletion of the gene uga2 coding for the succinate-semialdehyde dehydrogenase, is advantageous for an optimized production process for producing succinic acid. Glyoxylate, which is necessary for the glyoxylate cycle, cannot only be generated from the reaction catalyzed by the isocitrate lyase, wherein isocitrate is cleaved to succinate and glyoxylate, but also by the reaction of the alanine-glyoxylate aminotransferase. This enzyme catalyzes the generation of glyoxylate and alanine based on pyruvate and glycine. If glyoxylate must not necessarily be made available from the isocitrate lyase reaction, the glyoxylate cycle may also be secured by the reaction of the alanine-glyoxylate aminotransferase reaction, by which glyoxylate is provided. In this case, the isocitrate lyase activity, by which the intended product succinate is generated, is not necessary for the glyoxylate cycle, with the consequence that the yeast in part uses the alternative reaction catalyzed by the alanine-glyoxylate aminotransferase for the synthesis of glyoxylate. Then no succinate is generated, which would lead to yield losses. Therefore, the additional deletion of the gene agx1 coding for the alanine-glyoxylate aminotransferase is advantageous for an optimized production process for producing succinic acid.

The following observations must be made concerning feature c). In cellular respiration, carbon from the substrate, e.g., glucose, is not converted in ethanol or glycerol, as in fermentation, but enters into the respiratory central metabolism, i.e. into the citrate cycle or the glyoxylate cycle. When the carbon passes these two cycles, redox equivalents in the form of NADH or NADPH are generated, the electrons of which are transferred to the first protein complex of the respiratory chain, which is localized in the inner mitochondrial membrane. These electrons are then transferred step by step by further protein complexes of the respiratory chain to the final electron acceptor oxygen, which thereby is reduced to water. The released energy of this controlled oxyhydrogen reaction is used for transporting protons against a gradient into the intermembrane space of the mitochondria, which then when flowing back into the mitochondrion drive, by the complex V of the respiratory chain, the so-called “proton pump”, wherein energy in the form of ATP is generated. Under fermentation conditions, the yeast produces besides glycerol mainly ethanol and generates only 2 energy equivalents in the form of ATP per molecule glucose, compared to the respiration, in which 38 ATP can be generated per molecule glucose. In the fermentation, NADH is re-oxidized to NAD by the synthesis of ethanol or glycerol and not, as in respiration, by transfer of the electrons on oxygen, since under anaerobic conditions oxygen is not available as a final electron acceptor. The yeast Saccharomyces cerevisiae is “Crabtree” positive. This means that on primary carbon sources, such as for instance glucose, the yeast will even under aerobic conditions ferment and not respire. This fermentation activity can be observed already at a glucose concentration of approx. 100 mg/l glucose in the culture medium, since at this concentration the limit of the respiratory capacity of the yeast cell is reached. The reason for this is that in presence of glucose a multitude of the genes of the respiratory central metabolism, i.e. of the citrate and glyoxylate cycle (see FIG. 1a.) and b.)) and of the respiratory chain, are transcriptionally strongly suppressed (Gancedo 1998). This phenomenon is also called glucose or catabolite repression. The fermentation is another aspect, which may affect the biotechnological production of succinic acid, since it will lead to the generation of undesired side products. In this connection, mainly the generation of glycerol, acetate and ethanol poses problems, since it will lead to substantial yield losses. All organisms showing the Crabtree effect, such as for instance the yeast Saccharomyces cerevisiae, will ferment even under aerobic conditions. In the biotechnological production of succinic acid in yeast, the generation of fermentation end products, mainly ethanol, is in principle not undesired and avoidable. Under aerobic conditions, this can in part be achieved by that a continuous addition of a small amount of glucose to the culture medium is performed, which will prevent or reduce the glucose repression and thus the fermentation under aerobic conditions. Under anaerobic conditions, oxygen is not available as a final electron acceptor, thus the yeast in any case must ferment to reoxidize NADH and thus remain metabolically active. One possibility to prevent the alcoholic fermentation, i.e. the generation of ethanol, is the elimination of the ethanol biosynthesis based on pyruvate. For this purpose, the pyruvate decarboxylase activity can be turned off, which is catalyzed in the yeast Saccharomyces cerevisiae by 3 pyruvate decarboxylase isoenzymes, coded by the genes PDC1, PDC5 and PDC6. PDC6 is expressed very weakly only by the growth on glucose, as well as on ethanol (Velmurugan et al. 1997). The gene PDC2 codes for a transcriptional inductor, which is mainly responsible for the expression of the genes PDC1 and PDC5. The gene PDC2 offers thus the possibility to suppress in the cell with one single deletion only the main portion of the pyruvate decarboxylase activity coded by 3 genes. Of course, alternatively or additionally, one or more of the genes PDC1, PDC5 and/or PDC6 may also be deleted. This has the great advantage that thus the problematic ethanol generation can be prevented with one single modification only in the metabolism of the yeast. In order to minimize or reduce the yield losses in the biotechnological production of succinic acid in yeast, the generation of side products, mainly of ethanol, should be prevented or strongly reduced. This can be achieved by deletion of the gene PDC2 in the yeast Saccharomyces cerevisiae. Since this deletion leads to a strongly limited growth on glucose, a promoter that can suppressed or induced can be provided upstream of the gene PDC2. The promoter which can be suppressed secures a sufficient transcription of the gene provided downstream in the growth phase and stops the transcription in the production phase by an addition to the culture medium. The promoter which can be induced is induced during the growth phase by addition of an inductor to the culture medium, thereby the gene PDC2 provided downstream being sufficiently transcribed and a growth of the yeast culture being possible. When the inductor is consumed, no further growth is possible and the production phase is initiated. Without pyruvate decarboxylase activity, no growth of the yeast cell is possible, since no sufficient acetyl-CoA, generated by acetaldehyde and acetate, is available for the fatty acid biosynthesis.

The following observations must be made concerning feature d). Different enzymes of the citrate and glyoxylate cycle, the genes of which are transcriptionally suppressed by glucose, are subject also on the protein level to the regulation or inactivation by glucose. A transcriptional deregulation of these genes alone is therefore not sufficient to obtain to full extent an active gene product. For a method for producing succinic acid being optimized with regard to the production time and yields, inactivation effects on the protein level must therefore also be avoided. An example is one of the main enzymes of the glyoxylate cycle, the isocitrate lyase. This enzyme, which is essential for the efficient production of organic acids, is subject, in presence of glucose, to the inactivation by phosphorylation and an increased proteolytic degradation (Lopez-Boado et al. 1988; Ordiz et al. 1996). For further enzymes of the respiratory system, in particular of the glyoxylate cycle, glucose-induced negative regulatory effects on the protein level can also be assumed. This mainly relates to the acetyl-CoA synthetase (Acs1p), the malate synthase (Mls1p) and the malate dehydrogenase (Mdh3p). The glucose-induced proteolytic degradation or inactivation of these enzymes can be prevented by that heterologous isoenzymes are expressed in the yeast Saccharomyces cerevisiae. These enzymes originate from a microorganism, which naturally comprises a glyoxylate cycle and is “Crabtree”-negative, since enzymes of the glyoxylate cycle from such an organism are not subject to the glucose-induced negative regulation or the proteolytic degradation on the protein level. An active glyoxylate cycle is essential for an efficient production of organic acids with high yields on primary carbon sources. “Crabtree”-negative donor organisms are for instance Escherichia coli, Anaerobiospirillum, Actinobacillus, Mannheimia or Corynebacterium.

The term deletion of a gene designates the complete removal of the gene from the genome of the microorganism and/or the removal of the active enzyme coded thereby from the microorganism. An inactivation of a gene designates the reduction or the complete elimination of the activity of the enzyme or protein coded by the gene. This can be verified by a measurement of the respective enzymatic activity by means of conventional standard tests or by a determination of the respective enzyme or protein by means of for instance immunological detection reactions. An inactivation may for instance take place by reduction or inhibition of the gene expression (transcription and/or translation). For this purpose, for instance the introduction of antisense nucleic acids (by addition or by insertion of nucleic acid sequences into the genome, which can be transcribed to the antisense nucleic acid), the introduction of mutations into the endogene, which reduce or completely eliminate the activity of the gene product, the introduction of gene-specific DNA-binding factors, for instance zinc finger transcription factors, which cause a reduction of the gene expression, the replacement of the endogene by a foreign gene, which codes for a corresponding, however inactivated or less active enzyme or protein. Further, a promoter, under the control of which the respective endogene is, can be deleted or mutated, so that a transcription is reduced or inhibited.

The sequences (nucleic acid sequences and/or amino acid sequences) of the genes or enzymes inactivated according to the invention or the mentioned promoters can be obtained under the following gene database numbers or are described in the following documents.

### SDH1: NC_{—}001143.7

### PDC2: NC_{—}001136.8

### IDP1: NC_{—}001136.8

### IDP2: NC_{—}001144.4

### IDP3: NC_{—}001146.6

### AGX1: NC_{—}001138.4

### UGA2: NC_{—}001134.7

### MLS1: X64407 S50520

### ICL1: X65554

### MDH3: M98763

### ACS1: AY723758

aceA: NC_{—}000913.2

### acs: NC_{—}004431.1

aceB: NC_{—}010473.1

### mdh: NC_{—}000913.2

ADH1: Lang C., Looman A. C., Appl Microbiol biotechnol. 44(1-2): 147-156 (1995)
tetO and tTA: Gari E. et al., Yeast 13: 837-848 (1997).

The transformation of microorganisms, such as yeast cells, can be carried out in a conventional way and with respect thereto reference is made to the documents Schiestl R. H., et al., Curr Genet. December, 16(5-6):339-346 (1989), or Manivasakam P., et al., Nucleic Acids Res. September 11, 21(18):4414-4415 (1993), or Morgan A. J., Experientia Suppl., 46: 155-166 (1983).

Vehicles being suitable for the transformation, in particular plasmids, are for instance known from the documents Naumovski L., et al., J. Bacteriol. 152(1):323-331 (1982), Broach J. R., et al., Gene, 8(1):121-133 (1979), Sikorski R. S., et al., Genetics, 122(1)19-27 (1989). These vectors are Yep24, Yep13, pRS vector series, and YCp19 or pYEXBX.

The production of expression cassettes suitable for the purpose of the invention typically takes place by fusion of the promoter with the nucleic acid sequence coding for the gene and if applicable a terminator by conventional recombination and cloning techniques, as for instance described in the documents Maniatis T., et al., Molecular Cloning: A Laboratory Manual, Cold Spring Harbor Laboratory, Cold Spring Harbor, N.Y., USA, 1989, or Sihlavy T. J., et al., Experiments with Gene Fusions, Cold Spring Harbor Laboratory, Cold Spring Harbor, N.Y., USA, 1984, or Ausubel F. M., et al., Current Protocols in Molecular Biology, Greene Publishing Assoc. and Wiley-Interscience, 1987.

The invention further relates to the use of a microorganism according to the invention for producing an organic carboxylic acid of the glyoxylate and/or citrate cycle, in particular an organic dicarboxylic acid, preferably succinic acid, and to the use thereof in a method for producing an organic carboxylic acid of the glyoxylate and/or citrate cycle, in particular an organic dicarboxylic acid, preferably succinic acid, comprising the following steps: A) in a growth step, the microorganism is cultivated and multiplied under preferably aerobic conditions, optionally under addition of an inductor substance for inducing the promoter which can be induced and/or glutamate, B) then the microorganism is cultivated in a production phase under preferably anaerobic conditions, optionally under addition of an inductor substance for suppressing the promoter which can be suppressed, C) then after step B) or during step B), the carboxylic acid is separated from the culture supernatant and optionally purified.

In the method according to the invention it is preferred that step A) is carried out until reaching a cell density of at least 100 g dry biomass/l, preferably at least 120 g/l, most preferably at least 140 g/l. Step B) can be carried out until reaching a carboxylic acid concentration of at least 0.4 mole/l, preferably at least 0.8 mole/l, most preferably at least 1.0 mole/l. In step A), a pH in the range from 4 to 9, preferably from 6 to 8, and a salt concentration in the range from 0.01 to 0.5 mole/l, preferably from 0.05 to 0.2 mole/l, most preferably from 0.05 to 0.1 mole/l, can be adjusted. In step B), a pH in the range from 4 to 9, preferably from 6 to 8, and a salt concentration in the range from 0.01 to 0.5 mole/l, preferably from 0.05 to 0.2 mole/l, most preferably from 0.05 to 0.1 mole/l, can be adjusted. Step A) is preferably carried out at a temperature from 20 to 35° C., most preferably from to 30° C., and for a time from 1 to 1,000 h, preferably from 2 to 500 h, most preferably from 2 to 200 h. In step B), a temperature from 15 to 40° C., preferably from 20 to 35° C., most preferably from 28 to 30° C., and a time from 1 to 1,000 h, preferably from 2 to 500 h, most preferably from 2 to 200 h, is preferred.

As culture medium for step A) can for instance be used WMVIII-medium (Lang C., Looman A. C., Appl Microbiol Biotechnol. 44(1-2):147-156 (1995)). The amount of tetracycline in the culture medium is preferably below 20 mg/l, more preferably below 10 mg/l, most preferably below 1 mg/l, down to values that are below the detection limit, and/or the CuSO₄ concentration in the culture medium, if used, is preferably above 1 μM, most preferably above 5 μM. Ranges can for instance be 1 to 3 μM or 3 to 15 μM.

As culture medium for step B) can for instance be used WMVIII-medium, but a conventional molasse medium can also be used. The amount of tetracycline, if used, is preferably above 1 mg/l, most preferably above 3 mg/l. Ranges can for instance be 1 to 3 mg/l or 3 to 15 mg/l. The used CuSO₄ concentration in the culture medium is preferably below 20 μM, more preferably below 10 μM, most preferably below 1 μM, down to values that are below the detection limit.

Step C) then be carried out after step B). Then the culture supernatant is separated from the microorganisms, for instance by filtration or centrifugation. Step C) can however also be carried out during step B, and that continuously or discontinuously. In the latter case, at least part of the culture supernatant is removed and replaced by new culture medium, and this process is repeated several times, if applicable. From the removed culture supernatant, the succinic acid is obtained. A continuous separation can take place by suitable membranes or by conducting a flow of the culture mediums over a device for separating the succinic acid.

The invention further relates to a method for producing a microorganism according to the invention, wherein a) the genes idh1 and idp1 are deleted or inactivated, and/or b) the genes sdh2 and sdh1 are deleted or inactivated, and/or c) the gene PDC2 is deleted or inactivated or is under the control of a promoter which can be suppressed or induced by exposure of the microorganism using an inductor substance, and/or d) one or more genes from the group consisting of ICL1, MLS1, ACS1 and MDH3 are replaced or supplemented by a corresponding foreign gene or corresponding foreign genes from a Crabtree-negative organism.

In principle, all explanations given with regard to microorganisms according to the invention also apply in an analogous manner to the uses and methods according to the invention.

The bibliographic details for the documents mentioned above and below as short-form citations are as follows:

Contreras-Shannon, V., A. P. Lin, M. T. McCammon and L. McAlister-Henn (2005). “Kinetic properties and metabolic contributions of yeast mitochondrial and cytosolic NADP+-specific isocitrate dehydrogenases.” J Biol Chem 280(6):4469-75.

DeRisi, J. L., V. R. Iyer and P. O. Brown (1997). “Exploring the metabolic and genetic control of gene expression on a genomic scale.” Science 278(5338):680-6.

Gancedo, J. M. (1998). “Yeast carbon catabolite repression.” Microbiol Mol Biol Rev 62(2): 334-61.

Guldener, U., S. Heck, T. Fielder, J. Beinhauer and J. H. Hegemann (1996). “A new efficient gene disruption cassette for repeated use in budding yeast.” Nucleic Acids Res 24(13):2519-24.

Kubo, Y., H. Takagi and S, Nakamori (2000). “Effect of gene disruption of succinate dehydrogenase on succinate production in a sake yeast strain.” J Biosci Bioeng 90(6):619-24.

Lang, C. and A. C. Looman (1995). “Efficient expression and secretion of Aspergillus niger RH5344 polygalacturonase in Saccharomyces cerevisiae.”Appl Microbiol Biotechnol 44(1-2):147-56.

Lopez-Boado, Y. S., P. Herrero, T. Fernandez, R. Fernandez and F. Moreno (1988). “Glucose-stimulated phosphorylation of yeast isocitrate lyase in vivo.” J Gen Microbiol 134(9):2499-505.

Ordiz, I., P. Herrero, R. Rodicio and F. Moreno (1996). “Glucose-induced inactivation of isocitrate lyase in Saccharomyces cerevisiae is mediated by the cAMP-dependent protein kinase catalytic subunits Tpk1 and Tpk2.” FEBS Lett 385(1-2):43-6.

Velmurugan, S., Z. Lobo and P. K. Maitra (1997). “Suppression of pdc2 regulating pyruvate decarboxylase synthesis in yeast.” Genetics 145 (3):587-94.

The figures are intended for explaining the various synthesis ways described above and the modification thereof by genetic measures of the invention. There are:

FIG. 1: a representation of the citrate and glyoxylate cycle with the involved genes, metabolites and enzymes or proteins, and

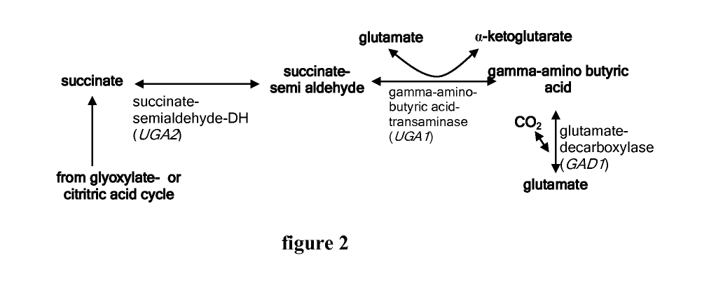

FIG. 2: metabolization of succinate to glutamate in the wild-type.

In the following, the invention is explained in more detail with reference to different examples. Individual features according to the invention are described with regard to microorganisms being exemplary only. The described genetic measures can of course also be transferred to other microorganisms, in particular yeasts. Further, the various genetic measures can also be provided in other combinations.

### Example1

### Production of a Microorganism Using a Glyoxylate Cycle, which Transcriptionally and on the Protein Level is not Subject to the Glucose Repression for Producing Organic Acids of the Respiratory Central Metabolism, in Particular of Succinic Acid

Different enzymes of the citrate and glyoxylate cycle, the genes of which are transcriptionally suppressed by glucose, are subject in the yeast Saccharomyces cerevisiae also on the protein level to the regulation or inactivation by glucose. A transcriptional deregulation of these genes is therefore not sufficient for obtaining an active gene product. Thus, for a method for producing succinic acid being optimized with regard to production time and yields, inactivation effects on the protein level must also be prevented.

The glucose-induced proteolytic degradation or inactivation of the enzymes of the glyoxylate cycle can be prevented by expressing heterologous isoenzymes in the yeast Saccharomyces cerevisiae. These enzymes originate from a microorganism, which naturally comprises a glyoxylate cycle and is “Crabtree”-negative, since enzymes of the glyoxylate cycle from such an organism are not subject to the negative regulation induced by glucose, or to the proteolytic degradation on the protein level. Donor organisms may for instance be Escherichia coli, Anaerobiospirillum, Actinobacillus, Mannheimia and Corynebacterium. The transcriptional deregulation of these enzymes is achieved by putting the corresponding genes under the control of a constitutive promoter.

For this purpose, the genes acs (acetyl-CoA synthetase), aceA (isocitrate lyase), aceB (malate synthase A), mdh (malate dehydrogenase) are amplified by means of PCR from bacterial DNA of the strain E. coli JM109 and provided with restriction linkers and then integrated in the yeast chromosome under the control of the constitutive ADH1 promoter. For the deregulated expression of this gene in the yeast Saccharomyces cerevisiae, the constitutive ADH1 promoter is used that leads by modification of the natural sequence over a very long time to a constitutive expression being independent from glucose and ethanol (Lang and Looman 1995).

The coding nucleic acid sequence for the expression cassette from ADH1prom-acs(aceA, aceB, mdh)-TRP1term. was amplified by PCR using standard methods from the vector pFlat1-acs(aceA, aceB, mdh). The obtained DNA fragment was blunt-end cloned after a Klenow treatment into the vector pUG6 in the EcoRV interface and resulted in the vector pUG6-acs(aceA, aceB, mdh). After plasmid isolation, an expanded fragment was amplified by means of PCR from the vector pUG6-acs(aceA, aceB, mdh), so that the resulting fragment consists of the following components: loxP-kanMX-loxP-ADH1-prom-acs(aceA, aceB, mdh)-tryptophan terminator. As primers were chosen oligonucleotide sequences that contain at the 5′ and 3′ overhangs respectively the 5′ or the 3′ sequence of the acs (aceA, aceB, mdh) gene and in the annealing section the sequences 5′ of the loxP regions and 3′ of the tryptophan terminator. Thus it is secured that on the one hand, the complete fragment including KanR and acs (aceA, aceB, mdh) is amplified and on the other hand, this fragment can then be transformed into yeast and integrates this complete fragment by homologous recombination into the corresponding gene locus of the yeast.

As a selection marker serves the respective resistance against G418. In order to then remove again the resistance against G418, the respective formed yeast strain is transformed with the cre recombinase vector pSH47 (Guldener et al. 1996). By this vector, the cre recombinase in the yeast is expressed, with the consequence that the sequence region within the two loxP sequences recombines out. The consequence is that only one of the two loxP sequences and the respective expression cassette remains contained in the original corresponding gene locus. The consequence is that the yeast strain loses again the G418 resistance and is thus suitable to integrate or to remove further genes by means of this cre-lox system in the yeast strain. The vector pSH47 can then be removed again by a counter selection on YNB agar plates supplemented with uracil (20 mg/L) and FOA (5-fluoroorotic acid) (1 g/L). For this purpose, the cells carrying this plasmid must first be cultivated under non-selective conditions and then be drawn on FOA-containing selective plates. Under these conditions, only those cells can grow that are not themselves capable of synthesizing uracil. These are in this case cells that do not contain any plasmid (pSH47) anymore.

### Example2

### Production of a Microorganism for the Biotechnological Production of Succinic Acid and Other Organic Acids, which makes a More Efficient Production Process Possible by Reduction of Yield Losses

In order to reduce yield losses during the biotechnological production of succinic acid in the yeast Saccharomyces cerevisiae, succinic acid must be enriched as an end product and must not be further metabolized by the yeast cell. This cannot be achieved to full extent by the singular deletion of the gene sdh2. In addition, another subunit of the heterotetrameric enzyme succinate dehydrogenase, coded by the gene sdh1, must be deleted.

Yield losses may also result from the further metabolization of the formed succinates by the enzyme succinate-semialdehyde dehydrogenase. This enzyme is part of the glutamate degradation pathway and catalyzes the reaction of succinate to succinate-semialdehyde. This intermediate is then metabolized by gamma-amino butyric acid to glutamate. In this way, not only yield losses may occur, but α-ketoglutarate and glutamate may also be synthesized, which makes a control of the fermentation process by glutamate supplementation impossible. Therefore, the additional deletion of the gene uga2 coding for the succinate-semialdehyde dehydrogenase is advantageous for an optimized production process for producing succinic acid.

Glyoxylate being necessary for the glyoxylate cycle cannot only occur from the reaction catalyzed by the isocitrate lyase, wherein isocitrate cleaves into succinate and glyoxylate, but also by the reaction of the alanine-glyoxylate aminotransferase. This enzyme catalyzes the generation of glyoxylate and alanine based on pyruvate and glycine. If glyoxylate is not necessarily provided by the isocitrate lyase reaction, the glyoxylate cycle can also be secured by the reaction of the alanine-glyoxylate aminotransferase reaction, by which glyoxylate is provided. In this case, the isocitrate lyase activity, by which the intended product succinate is generated, is not necessary for the glyoxylate cycle, with the consequence that the yeast in part uses the alternative reaction catalyzed by the alanine-glyoxylate aminotransferase for the glyoxylate synthesis. Therein, no succinate, which could lead to yield losses, is generated. Therefore, the additional deletion of the gene agx1 coding for the alanine-glyoxylate aminotransferase is advantageous for an optimized production process for producing succinic acid.

The idh1 deletion does not lead to a complete disappearance of the isocitrate dehydrogenase activity. The reason for this is 3 further isoenzymes of the isocitrate dehydrogenase, which can compensate the omission of the dimeric main enzyme, coded by the genes IDH1 and IDH2, with respect to the generation of α-ketoglutarate. In addition to the deletion of the gene idh1, at least the gene idp1 coding for an isoenzyme of the isocitrate dehydrogenase must also be deleted, in order to completely prevent the isocitrate dehydrogenase activity on glucose. The complete inhibition of the isocitrate dehydrogenase activity has the advantage that all carbon in the respiratory system of the yeast is redirected into the glyoxylate cycle in the direction of succinic acid and cannot flow off to α-ketoglutarate, which would lead to yield losses.

In brief, yield losses during the biotechnological production of succinic acid in yeast can be minimized or reduced by that the genes sdh1, agx1, uga2 and idp1 are deleted in addition to the genes sdh2 and idh1.

For this purpose, the coding nucleic acid sequence for the deletion cassette loxP-kanMX-loxP was amplified from the vector pUG6 by PCR using standard methods (Guldener et al. 1996), so that the resulting fragment consists of the following components: loxP-kanMX-loxP. As primers were chosen oligonucleotide sequences that contain at the 5′ and 3′ overhangs respectively the 5′ or the 3′ sequence at the beginning and at the end of the native locus of the genes to be deleted (sdh1, agx1, uga2, idp1) and in the annealing region the sequences 5′ of the loxP region and 3′ of the second loxP Region. Thus it is secured that on the one hand the complete fragment loxP-kanMX-loxP is amplified and on the other hand this fragment can then be transformed into yeast and integrates by homologous recombination this complete fragment into the gene locus to be deleted of the yeast.

As a selection marker serves the resistance against G418 (coded by kanMX). In order to then remove again the resistance against G418 and to allow a further use of the kanMX marker, the formed yeast strain is transformed with the cre recombinase vector pSH47 (Guldener et al. 1996). By this vector, the cre recombinase in the yeast is expressed, with the consequence that the sequence region within the two loxP sequences recombines out. The consequence is that only one of the two loxP sequences remains at the deleted gene locus (sdh1, agx1, uga2, idp1). The consequence is that the yeast strain loses again the G418 resistance and is thus suitable to integrate or to remove further genes by means of this cre-lox system in the yeast strain. The vector pSH47 can then be removed again by a counter selection on YNB agar plates supplemented with uracil (20 mg/L) and FOA (5-fluoroorotic acid) (1 g/L). For this purpose, the cells carrying this plasmid must first be cultivated under non-selective conditions and then be drawn on FOA-containing selective plates. Under these conditions, only those cells can grow that are not themselves capable of synthesizing uracil. These are in this case cells that do not contain any plasmid (pSH47) anymore. In this way, all genes to be deleted (sdh1, agx1, uga2, idp1) were iteratively deleted.

Table 1 exemplarily shows the yield increases by the above deletions after cultivation of the strains mentioned in the Table for 72 hours in WM8 medium (Lang and Looman, 1995) with 3.52 g/l ammonium sulfate and 0.05 M Na₂HPO₄ and 0.05 M NaH₂HPO₄ as buffer, and with histidine 100 mg/l, leucine 400 mg/l, uracil 100 mg/l. The C source was 5% glucose. An inoculation was made with 1% from a h pre-culture. Cultivation was made in 100 ml shake flask on a shake incubator at 30° C. and 150 rpm.

Since strains 5 and 6 do not grow in medium without glutamate, first biomass was generated with these strains in 75 ml standard WM8 medium with Na glutamate and glucose 5% in 250 ml baffled flask. The cells were washed and resuspended in the above medium for further cultivation.

TABLE 1
Succinate titer after 72 hours cultivation of the mentioned
strains in WM8 medium under the above conditions.

| | | Succinate in the culture |
|---|---|---|
| | Strain | supernatant in g/l |
| 1 | AH22ura3 (wild type) | 0.12 |
| 2 | AH22ura3Δsdh2Δidh1 | 0.41 |
| 3 | AH22ura3Δsdh2Δsdh1Δidh1Δidp1 | 2.5 |

In Table 1 can be seen that all performed deletions lead a higher succinate quantity in the culture supernatant, compared to the wild type. The quadruplet deletion mutant AH22ura3Δsdh2Δsdh1Δidh1Δidp1 enriches the highest amount of succinate. Corresponding results are also obtained with other microorganisms or yeasts.

### Example3

### Production of a Microorganism for the Biotechnological Production of Succinic Acid and Other Organic Acids, which makes a More Efficient Production Process Possible by Reduction of Side Product Generation, in Particular of Ethanol and Acetate

Fermentation is another essential aspect, which is disadvantageous for the biotechnological production of succinic acid, since it leads to the generation of undesired side products. In this connection, mainly the generation of acetate and ethanol poses problems, since it leads to substantial yield losses.

One possibility to prevent the alcoholic fermentation, i.e. the generation of ethanol, is the elimination of the ethanol biosynthesis based on pyruvate. Thereby, the acetate generation by acetaldehyde is also prevented. For this purpose, the pyruvate decarboxylase activity must be turned off, which is catalyzed in the yeast Saccharomyces cerevisiae by 3 pyruvate decarboxylase isoenzymes, coded by the genes PDC1, PDC5 and PDC6. The gene PDC2 codes for a transcriptional inductor, which is mainly responsible for the expression of the genes PDC1 and PDC5. Thus, the gene PDC2 offers the possibility to eliminate with one single deletion only the main portion of the pyruvate decarboxylase activity coded by 3 genes in the cell. This has the great advantage that thereby the problematic ethanol generation can be prevented with a single modification only in the metabolism of the yeast.

This can be achieved by deletion of the gene PDC2 in the yeast Saccharomyces cerevisiae. Since this deletion leads to a strongly restricted growth on glucose, a promoter which can be induced can be connected upstream of the gene PDC2. The promoter which can be induced is induced during the growth phase by addition of an inductor to the culture medium, whereby the downstream gene PDC2 is sufficiently transcribed and a growth of the yeast culture is secured. When the inductor is consumed, no further growth is possible and the production phase without side product generation in the form of ethanol and acetate is initiated.

As promoter which can be induced, the CUP1 promoter was chosen and chromosomally integrated before the “open reading frame” of the gene PDC2, so that the latter is under the control of the CUP1 promoter which can be induced by copper.

For this purpose, the coding nucleic acid sequence for the CUP1 promoter cassette was amplified by PCR using standard methods, so that the resulting fragment consists of the following components: loxP-kanMX-loxP-CUP1pr. As primers, oligonucleotide sequences were chosen that contain at the 5′ and 3′ overhangs respectively the 5′ or the 3′ sequence of the native promoter of the PDC2 gene and in the annealing region the sequences 5′ of the loxP-Region and 3′ of the CUP1prom. Thus it is secured that on the one hand the complete fragment including kanMX and CUP1 promoter can be amplified, and on the other hand this fragment can then be transformed into yeast and integrates this complete fragment by homologous recombination into the PDC2 gene locus of the yeast, before the coding region of the gene PDC2.

As a selection marker serves the resistance against G418. The resulting strain contains a copy of the gene PDC2 under the control of the copper-regulated CUP1 promoter and of the native PDC2 terminator. In order to then remove again the resistance against G418, the formed yeast strain is transformed with the cre recombinase vector pSH47 (Guldener et al. 1996). By this vector, the cre recombinase in the yeast is expressed, with the consequence that the sequence region within the two loxP sequences recombines out. The consequence is that only one of the two loxP sequences and the CUP1 promoter cassette remains contained before the coding sequence of the gene PDC2. The consequence is that the yeast strain loses again the G418 resistance and is thus suitable to integrate or to remove further genes by means of this cre-lox system in the yeast strain. The vector pSH47 can then be removed again by a counter selection on YNB agar plates supplemented with uracil (20 mg/L) and FOA (5-fluoroorotic acid) (1 g/L). For this purpose, the cells carrying this plasmid must first be cultivated under non-selective conditions and then be drawn on FOA-containing selective plates. Under these conditions, only those cells can grow that are not themselves capable of synthesizing uracil. These are in this case cells that do not contain any plasmid (pSH47) anymore.

### Example3

### Production of a Microorganism for the Biotechnological Production of Succinic Acid and Other Organic Acids, which Makes a Separation of Growth and Production Phases by Glutamate Supplementation Possible

In the following, a possibility of the separation of growth and production phases, which does not require the use of antibiotics, is described. In the yeast Saccharomyces cerevisiae, in spite of the deletions of the genes sdh2 and idh1, which lead to an interruption of the citrate cycle (see FIG. 1 black crosses), a growth rate can be measured that is comparable to an unmodified wild type yeast (in the 100 ml shake flask in YPD-medium, the strain AH22ura3Δsdh2Δidh1 has a growth rate that is only by 11% smaller, compared to the strain AH22ura3 (wild type), source: own data).

A growth of a yeast strain with an idh1 deletion is possible only because this deletion does not lead to a complete disappearance of the isocitrate dehydrogenase activity. The reason for this are 3 further isoenzymes of the isocitrate dehydrogenase, which can compensate the omission of the dimeric main enzyme, coded by the genes IDH1 and IDH2, with respect to the generation of α-ketoglutarate. The synthesis of α-ketoglutarate is absolutely necessary for a growth of the yeast cell on minimal media, since from this intermediate the amino acid glutamate is generated, without which no growth is possible.

In a 2-step fermentation process for producing succinic acid with yeast, the effective separation of growth and production phases is thus only possible by the complete inhibition of the isocitrate dehydrogenase activity in the production strain, because thereby a glutamate auxotrophy is secured, with the consequence that the yeast in the medium has no growth without supplemented glutamate. This can be used for controlling the fermentation process by the supplementation of glutamate to the culture medium. By the amount of added glutamate to the culture medium, the time of the growth phase, and the intended cell density can effectively be controlled in this phase. With increasing quantity, duration and cell density will also increase.

When the glutamate in the culture medium is consumed, no further growth is possible and all carbon can effectively be used for the synthesis of succinic acid, the generation of which will then not be competing with the generation of biomass. This essentially contributes to the increase of the yield and of the efficiency of the production process. This separation of growth and production phases in the fermentation process on glucose and other fermentable carbon sources by the supplementation of glutamate can only be materialized, if in addition to the deletion of the gene idh1 at least the gene idp1 coding for an isoenzyme of the isocitrate dehydrogenase, is also deleted. Only the complete inhibition of the isocitrate dehydrogenase activity secures the necessary glutamate auxotrophy.

Another advantage of the complete inhibition of the isocitrate dehydrogenase activity is that thus all carbon in the respiratory system of the yeast is redirected into the glyoxylate cycle in the direction of succinic acid and cannot flow off to α-ketoglutarate, which would lead to yield losses (see FIG. 1 e.)).

For this purpose, the coding nucleic acid sequence for the deletion cassette loxP-kanMX-loxP was amplified from the vector pUG6 by PCR using standard methods (Guldener et al. 1996), so that the resulting fragment consists of the following components: loxP-kanMX-loxP. As primers were chosen oligonucleotide sequences that contain at the 5′ and 3′ overhangs respectively the 5′ or the 3′ sequence at the beginning and at the end of the native locus of the gene idp1 to be deleted and in the annealing region the sequences 5′ of the loxP region and 3′ of the second loxP Region. Thus it is secured that on the one hand the complete fragment loxP-kanMX-loxP is amplified and on the other hand this fragment can then be transformed into yeast and integrates by homologous recombination this complete fragment into the gene locus to be deleted of the yeast.

As a selection marker serves the respective resistance against G418 (coded by kanMX). In order to then remove again the resistance against G418 and to thus allow a further use of the kanMX marker, the formed yeast strain is transformed with the cre recombinase vector pSH47 (Guldener et al. 1996). By this vector, the cre recombinase in the yeast is expressed, with the consequence that the sequence region within the two loxP sequences recombines out. The consequence is that only one of the two loxP sequences remains at the deleted gene locus idp1. The consequence is that the yeast strain loses again the G418 resistance and is thus suitable to integrate or to remove further genes by means of this cre-lox system in the yeast strain. The vector pSH47 can then be removed again by a counter selection on YNB agar plates supplemented with uracil (20 mg/L) and FOA (5-fluoroorotic acid) (1 g/L). For this purpose, the cells carrying this plasmid must first be cultivated under non-selective conditions and then be drawn on FOA-containing selective plates. Under these conditions, only those cells can grow that are not themselves capable of synthesizing uracil. These are in this case cells that do not contain any plasmid (pSH47) anymore.

The production strain AH22ura3Δsdh1Δsdh2Δidh1Δidp1 was evaluated according to its growth properties with and without supplemented glutamate. As reference strain AH22ura3Δsdh1Δsdh2Δidh1, that is the strain without additional deletion of idp1, was taken.

The two strains were cultivated in 20 ml WM8 medium in a 100 ml flask with 3.52 g/l ammonium sulfate (as nitrogen source) and 0.05 M K₂HPO₄, and 0.05 M KH₂HPO₄ as buffer and in standard WM8 medium (Lang and Looman 1995), which contains 10 g Na glutamate as nitrogen source. After 64 h, the optical density of the 4 cultures was determined. The results are shown in Table 2.

TABLE 2
Optical density of the mentioned strains after 64 hours
cultivation in WM8 medium with and without glutamate.

| | | OD600/ml in | OD600/ml in |
|---|---|---|---|
| | | WM8 with | WM8 without |
| | Strain | Na glutamate | Na glutamate |
| 1 | AH22ura3Δsdh2Δsdh1Δidh1 | 26.4 | 10 |
| 2 | AH22ura3Δsdh2Δsdh1Δidh1Δidp1 | 29.5 | 0 |

In the medium without glutamate, NH₃SO₄ was supplemented as nitrogen source.

In Table 2 can be seen that the additional deletion of idp1 in an idh1 strain leads to a glutamate auxotrophy on glucose, since the strain AH22ura3Δsdh2Δsdh1Δidh1Δidp1 in the medium without glutamate shows no growth, other than AH22ura3Δsdh2Δsdh1Δidh1. The singular deletion of idh1 does not lead to a glutamate auxotrophy. The separation of growth and production phases in a 2-step production process for producing succinic acid and other organic acids on glucose by glutamate supplementation is only possible in a strain with the deleted genes idh1 and idp1. When another nitrogen source, such as for instance ammonium sulfate, is added, a growth of a Δidh1Δidp1 mutant is possible in minimal medium already at very low amounts of supplemented glutamate (approx. 20 mg/l).

### LEGEND OF THE FIGURES

### FIG. 1

Glucose
Glycolysis Glycerol aldehyde-3-phosphate Glycerol-3-phosphate h) Glycerol
Glycerol-3-phosphate-OH Glycerol phosphatase
Phosphoenolpyruvate
Pyruvate kinase
CO₂
Oxalacetate Pyruvate Acetaldehyde h) Ethanol
Pyruvate carboxylase (PYC1) Pyruvate decarboxylase Alcohol DH
Aldehyde DH
Pyruvate Acetate
Pyruvate DH Acetyl-CoA synthetase (ACS1)
Acetyl-CoA Acetyl-CoA
Oxalacetate Citrate synthase (CIT1, CIT2)
Oxalacetate
Citrate
Malate DH MDH1 b) Glyoxylate cycle Malate DH (MDH3)
Aconitase (ACO1)
Isocitrate Malate
Acetyl-CoA
Fumarase (FUM1) a) Citrate cycle e) Isocitrate DH (IDH1) c) CO₂ Glyoxylate Malate dehydrogenase (MDH3)
Fumarate α-Ketoglutarate Isocitrate lyase
Alanine Glycine
Fumarate reductase (OSA11) ? α-Ketogl. DH
Succinyl-CoA c) CO₂ Alanine glyoxylate aminotransferase (AGX1)
Succinate Succinate Pyruvate
Cytosol Mitochondrion Cytosol
NH₃ d) Glutamate DH
Glutamate

### FIG. 2

Glutamate α-Ketoglutarate
Succinate Succinate semialdehyde Gamma-amino butyric acid
Succinate semialdehyde DH (UGA2) Gamma-amino butyric acid transaminase CO₂ Glutamate decarboxylase (GAD1)
From glyoxylate or citrate cycle Glutamate

1. An isolated genetically modified microorganism, wherein compared to the wild type
a) the idh1 and idp1 genes have been deleted or inactivated, and/or
b) the sdh2 and sdh1 genes have been deleted or inactivated, and/or
c) the PDC2 gene has been deleted or inactivated or is under the control of a promoter which can be suppressed or induced by exposure of the microorganism using an inductor substance, and/or
d) one or more genes from the group consisting of ICL1, MLS1, ACS1 and MDH3 has been replaced or supplemented by a corresponding foreign gene or corresponding foreign genes from a Crabtree-negative organism.

2. The microorganism according to claim 1, wherein in addition to the genes idh1 and idp1, one of the genes idp2 or idp3, or both genes, is/are deleted or inactivated.

3. The microorganism according to claim 1 or 2, wherein in addition to the genes sdh2 and sdh1, one of the genes uga2 or agx1, or both genes, is/are deleted or inactivated.

4. The microorganism according to one of claims 1 to 3, wherein the promoter connected upstream of the gene PDC2 is a promoter which can be induced, for instance CUP1.

5. The microorganism according to one of claims 1 to 3, wherein the promoter connected upstream of the gene PDC2 is a promoter which can be suppressed, for instance the tetracycline-regulated tetO-promoter.

6. The microorganism according to one of claims 1 to 5, wherein the foreign gene originates from a Crabtree-negative organism from the group consisting of Escherichia coli, Anaerobiospirillum, Actinobacillus, Mannheimia, Rhyzopus Corynebacterium, Schizosaccharomyces, Wickerhamia, Debayomyces, Hansenula, Hanseniaspora, Pichia, Kloeckera, Candida, Ogataea, Kuraishia, Komagataella, Yarrowia, Metschnikowia, Williopsis, Nakazawaea, Kluyveromyces, Cryptococcus, Torulaspora, Bullera, Rhodotorula, Willopsis, Kloeckera, Trichosporon, Yamadazmya and Sporobolomyces.

7. The microorganism according to one of claims 1 to 6, wherein the foreign gene is under the control of a constitutively active promoter, for instance of the ADH1 promoter.

8. The microorganism according to one of claims 1 to 7, wherein the microorganism is a yeast, preferably selected from the group consisting of Saccharomyces cerevisiae, Saccharomyces, Saccharomycecopsis, Saccharomycodes, Schizosaccharomyces, Wickerhamia, Debayomyces, Hansenula, Hanseniaspora, Pichia, Kloeckera, Candida, Zygosaccharomyces, Ogataea, Kuraishia, Komagataella, Yarrowia, Metschnikowia, Williopsis, Nakazawaea, Kluyveromyces, Cryptococcus, Torulaspora, Bullera, Rhodotorula, Willopsis, Kloeckera and Sporobolomyces.

9. The use of a microorganism according to one of claims 1 to 8 for producing an organic carboxylic acid of the glyoxylate and/or citrate cycle, in particular an organic dicarboxylic acid, preferably succinic acid.

10. The use of a microorganism according to one of claims 1 to 8 in a method for producing an organic carboxylic acid of the glyoxylate and/or citrate cycle, in particular an organic dicarboxylic acid, preferably succinic acid, comprising the following steps:
A) in a growth step, the microorganism is cultivated and multiplied under preferably aerobic conditions, optionally under addition of an inductor substance for inducing the promoter which can be induced and/or glutamate,
B) then the microorganism is cultivated in a production phase under preferably anaerobic conditions, optionally under addition of an inductor substance for suppressing the promoter which can be suppressed,
C) then after step B) or during step B), the carboxylic acid is separated from the culture supernatant and optionally purified.

11. The use according to claim 10, wherein step A) is carried out until a cell density of at least 100 g dry biomass/l, preferably at least 120 g dry biomass/l, most preferably at least 140 g dry biomass/1 is reached.

12. The use according to one of claim 10 or 11, wherein step B) is carried out until a carboxylic acid concentration of at least 0.4 mole/l, preferably at least 0.8 mole/l, most preferably at least 1.0 mole/l is reached.

13. The use according to one of claims 10 to 12, wherein step A) is carried out at a temperature of to 35° C., preferably of 28 to 30° C., and for a time of 1 to 1,000 h, preferably 2 to 500 h, most preferably 2 to 200 h.

14. The use according to one of claims 10 to 13, wherein step B) is carried out at a temperature of to 40° C., preferably of 20 to 35° C., and for a time of 1 to 1,000 h, preferably 2 to 500 h, most preferably 2 to 200 h.

15. A method for producing a microorganism according to one of claims 1 to 8, wherein
a) the idh1 and idp1 genes are deleted or inactivated, and/or
b) the sdh2 and sdh1 genes are deleted or inactivated, and/or
c) the PDC2 gene is deleted or inactivated or is under the control of a promoter which can be suppressed or induced by exposure of the microorganism using an inductor substance, and/or
d) one or more genes from the group consisting of ICL1, MLS1, ACS1 and MDH3 is replaced or supplemented by a corresponding foreign gene or corresponding foreign genes from a Crabtree-negative organism.
